(19) United States
(12) Patent Application Publication
(10) Pub. No.: US 2023/0349904 A1
(43) Pub. Date: Nov. 2, 2023

(54) METHODS FOR DETECTING ANTIBODIES

(71) Applicant: The Regents of the University of California, Oakland, CA (US)

(72) Inventor: Xianjin Zhou, La Jolla, CA (US)

(21) Appl. No.: 18/029,467

(22) PCT Filed: Oct. 5, 2021
(86) PCT No.: PCT/US2021/053525

§ 371 Date: Mar. 30, 2023

Related U.S. Application Data
(60) Provisional application No. 63/088,025, filed on Oct. 6, 2020.

(51) Int. Cl. G01N 33/569

(52) U.S. Cl. CPC G01N 33/56983; G01N 2333/165; G01N 2469/20

(57) ABSTRACT

Provided herein are methods of preparing a detectable antibody-antibody binding agent aggregate, the method comprising: (a) contacting a biological sample with an antibody binding agent, wherein the antibody binding agent comprises an antibody binding antigen and a detectable label, wherein the antibody binding agent binds an antibody in the biological sample; (b) adding an aggregating agent to the biological sample, wherein the aggregating agent binds to the antibody and forms an antibody-antibody binding agent aggregate; and (c) detecting a signal from the detectable label of the antibody binding agent associated with the antibody-antibody binding agent aggregate, thereby preparing a detectable antibody-antibody binding agent aggregate.

### CROSS-REFERENCE TO RELATED APPLICATIONS

This application claims priority to U.S. Application Ser. No. 63/088,025, filed on Oct. 6, 2020. The disclosure of the prior application is considered part of the disclosure of this application, and is incorporated in its entirety into this application.

### SEQUENCE LISTING

This application contains a Sequence Listing that has been submitted electronically as an ASCII text file named 15670-0348WO1_ST25.txt. The ASCII text file, created on Oct. 1, 2021, is 19.3 kilobytes in size. The material in the ASCII text file is hereby incorporated by reference in its entirety.

### BACKGROUND

Detection of antibodies is a useful tool in the diagnosis of diseases or determining the risk of a patient developing a disease. A simple, sensitive, and fast detection of antibodies against an antigen (e.g., SARS-COV2 virus) in blood or body fluid is essential for point-of care, high-throughput screening in research, and self-examination of antibodies by individuals.

### SUMMARY

Provided herein are methods of preparing a detectable antibody-antibody binding agent aggregate, the method comprising: (a) contacting a biological sample with an antibody binding agent, wherein the antibody binding agent comprises an antibody binding antigen and a detectable label, wherein the antibody binding agent binds an antibody in the biological sample; (b) adding an aggregating agent to the biological sample, wherein the aggregating agent binds to the antibody and forms an antibody-antibody binding agent aggregate; and (c) detecting a signal from the detectable label of the antibody binding agent associated with the antibody-antibody binding agent aggregate, thereby preparing a detectable antibody-antibody binding agent aggregate.

In some embodiments, the antibody is an IgG antibody. In some embodiments, the antibody is a polyclonal antibody. In some embodiments, the antibody is a monoclonal antibody. In some embodiments, the antibody is a SARS-COV2 antibody. In some embodiments, the antibody is generated against a full-length antigen. In some embodiments, the antibody is generated against an antigenic epitope.

In some embodiments, the biological sample is a serum. In some embodiments, the biological sample is nose mucus. In some embodiments, the serum is from an animal. In some embodiments, the serum is from a human.

In some embodiments, the antibody binding antigen is a SARS-COV2 protein. In some embodiments, the SARS-COV2 protein is a SARS-COV2 N nucleocapsid protein. In some embodiments, the SARS-COV2 protein is a SARS-COV2 spike protein.

In some embodiments, the detectable label comprises a fluorophore. In some embodiments, the fluorophore comprises a green fluorescent protein (GFP). In some embodiments, the detectable label comprises a bioluminescent label. In some embodiments, the bioluminescent label comprises Gaussia luciferase (Gluc). In some embodiments, the method further comprises adding a substrate of the bioluminescent label to the biological sample. In some embodiments, the detectable label is conjugated with the antibody binding antigen via cross-linking. In some embodiments, the detectable label is conjugated with the antibody binding antigen via GFP-complementation.

In some embodiments, the aggregating agent comprises a fusion protein. In some embodiments, the fusion protein is a protein-A/G/L fusion protein. In some embodiments, the fusion protein comprises protein-A, protein-G, protein-A/G, or variants thereof.

In some embodiments, the detecting comprises imaging the biological sample. In some embodiments, the imaging comprises fluorescent microscopy. In some embodiments, the imaging comprises using 470 nm blue LED light.

In some embodiments, the antibody binding agent is produced by expressing the antibody binding agent in a cell. In some embodiments, the cell is an E. coli cell.

In some embodiments, the antibody-antibody binding agent aggregate is detected within 5 minutes of adding the aggregating agent to the biological sample. In some embodiments, the antibody-antibody binding agent aggregate can be detected up to 5 days after adding the aggregating agent to the biological sample.

Also provided herein are kits comprising: (a) an antibody binding agent, wherein the antibody binding agent comprises an antibody binding antigen and a detectable label; (b) an aggregating agent; and (c) instructions to add the antibody binding agent and the aggregating agent to a biological sample to generate an antibody-antibody binding agent aggregate.

In some embodiments, the antibody binding antigen is a SARS-COV2 protein. In some embodiments, the SARS-COV2 protein is a SARS-COV2 N nucleocapsid protein. In some embodiments, the SARS-COV2 protein is a SARS-COV2 spike protein.

In some embodiments, the detectable label comprises a fluorophore. In some embodiments, the fluorophore comprises a green fluorescent protein (GFP). In some embodiments, the detectable label comprises a bioluminescent label. In some embodiments, the bioluminescent label comprises Gaussia luciferase (Gluc). In some embodiments, the detectable label is conjugated with the antibody binding antigen via cross-linking. In some embodiments, the detectable label is conjugated with the antibody binding antigen via GFP-complementation.

In some embodiments, the aggregating agent comprises a fusion protein. In some embodiments, the fusion protein is a protein-A/G/L fusion protein. In some embodiments, the fusion protein comprises protein-A, protein-G, protein-A/G, or variants thereof.

In some embodiments, the instructions further comprise instructions to image the biological sample.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 is an exemplary fluorescent image showing detection of anti-SARS-COV2 N antibodies, wherein different amounts of the anti-SARS-COV2 N antibodies (0.1 μg, 0.01 μg, and 0.001 μg) were incubated with the GFP-N fusion proteins.

FIG. 2 is an exemplary fluorescent image showing robustness of the detection of anti-SARS-COV2 N antibodies, wherein anti-SARS-COV2 antibodies were incubated with GFP-N fusion proteins for 5 minutes and 5 days.

FIG. 3 is an exemplary fluorescent image showing detection of anti-SARS-COV2 neutralizing antibodies against the receptor binding domain (RBD) of the spike protein in blood from pre-pandemic human sera from San Diego Blood Bank (SDBB) (left panel, SDBB1 to SDBB5) and sera from recovered COVID19 patients (right panel, P1 to P5).

FIG. 4 is an exemplary fluorescent image showing detection of anti-SARS-COV2 neutralizing antibodies against RBD of the spike protein in serum from subjects that were vaccinated with vaccines from either Pfizer or Moderna.

FIG. 5 is an exemplary fluorescent image showing detection of anti-SARS-COV2 neutralizing antibodies against RBD of the spike protein from both serum and nose mucus from a subject that was vaccinated with the Moderna vaccine three weeks after the first dose (upper panel) and two weeks after the second dose (lower panel).

FIG. 6 is an exemplary fluorescent image showing detection of anti-NMDAR1 antibodies against NMDAR1 P2 antigenic epitope in mouse blood serum.

FIG. 7A is an exemplary schematic showing a strategy of direct visualization of antigen (NMRAD1)-antibody (anti-NMDAR antibody) reaction via aggregation by protein A/G/L wherein the antigen is labeled with GFP. Protein A/G/L cross-links with the antibodies that bind antigen-GFP to form high fluorescence aggregates.

FIG. 7B is an exemplary fluorescent image showing detection of anti-NMDAR1 antibodies, wherein four mice were immunized with Complete Freund Adjuvant (CFA) only (left panel) and four mice were immunized with both CFA and NMDAR1 P2 peptide. The group of mice that were immunized with both CFA and NMDAR1 P2 peptide produced anti-NMDAR1 autoantibodies that were detected as protein aggregates.

FIG. 8A is an exemplary image showing SARS-COV2 N nucleocapsid protein-Gaussia luciferase (Gluc) fusion protein having strong luciferase activities by emitting blue light when incubated with substrate coelenterazine.

FIG. 8B is an exemplary image showing Gluc-N fusion proteins incubated with negative control serum (N) and serum from a COVID19 patient (P), wherein the P spot is shown to be positive for anti-SARS_COV2 N antibodies by emitting blue light.

FIG. 8C is an exemplary image showing Gluc-N fusion proteins incubated with negative control serum (N) and serum from a COVID19 patient (P), wherein the P spot is shown to be positive for anti-SARS_COV2 N antibodies by emitting blue light in the dark.

FIG. 9A is an exemplary schematic of a strategy of direct visualization of antigen-antibody reaction via aggregation by protein A/G/L. The antigen is labeled with GFP and protein A/G/L cross-links Ig Fc and/or light chain of all antibodies (IgG, IgM, IgA, IgE, and IgD) that bind antigen-GFP to form high fluorescence aggregates. Aggregating fluorescent antigen-antibody complexes depletes background fluorescence to achieve a high sensitivity.

FIG. 9B is an exemplary schematic of a one-step assay where rabbit polyclonal antibodies against SARS-CoV2 N nucleocapsid proteins were diluted in human serum and incubated with the scN-GFP fusion proteins for 5 minutes to examine the detection of aggregated antibodies.

FIG. 9C is an exemplary fluorescent image showing detection of anti-SARS-COV2 N antibodies, wherein 100 ng of the anti-SARS-COV2 N antibodies was incubated with the GFP-N fusion proteins for 5 min to examine the detection of antibody aggregates.

FIG. 9D is an exemplary fluorescent image showing detection of anti-SARS-COV2 N antibodies, wherein 10 ng of the anti-SARS-COV2 N antibodies was incubated with the GFP-N fusion proteins for 5 min to examine the detection of antibody aggregates.

FIG. 9E is an exemplary fluorescent image showing detection of anti-SARS-COV2 N antibodies, wherein 1 ng of the anti-SARS-COV2 N antibodies was incubated with the GFP-N fusion proteins for 5 min to examine the detection of antibody aggregates.

FIG. 9F is an exemplary fluorescent image showing detection of anti-SARS-COV2 N antibodies, wherein negative control serum was incubated with the GFP-N fusion proteins for 5 days to examine the detection of antibody aggregates.

FIG. 9G is an exemplary fluorescent image showing detection of anti-SARS-COV2 N antibodies, wherein 100 ng of the anti-SARS-COV2 N antibodies was incubated with the GFP-N fusion proteins for 5 days to examine the detection of antibody aggregates.

FIGS. 10A-10O are exemplary fluorescent images showing detection of anti-SARS-COV2 N antibodies. Sensitivity and specificity of the one-step assay in patient serum samples are shown here, wherein the positive control was human serum containing 100 ng of the rabbit antibodies against SARS-CoV2 N nucleocapsid protein (FIG. 10A); the negative controls are 4 representative samples (SDBB1-4 (FIGS. 10B-10E)) from the 50 pre-pandemic human serum samples from San Diego Blood Bank (SDBB); Patient Group A used SARS-CoV2 patient sera deemed positive (either IgG or IgM positive) and all of the patient samples were positive for anti-SARS-COV2 N antibodies (FIGS. 10F-10J); Patient Group B used SARS-CoV2 patient serum samples deemed negative (both IgG and IgM are negative) and the negative samples were positive for anti-SARS-COV2 N antibodies (FIGS. 10K-100).

### DETAILED DESCRIPTION

This disclosure describes methods of preparing a detectable antibody-antibody binding agent aggregate including (a) contacting a biological sample with an antibody binding agent, wherein the antibody binding agent comprises an antibody binding antigen and a detectable label, wherein the antibody binding agent binds an antibody in the biological sample; (b) adding an aggregating agent to the biological sample, wherein the aggregating agent binds to the antibody and forms an antibody-antibody binding agent aggregate; and (c) detecting a signal from the detectable label of the antibody binding agent associated with the antibody-antibody binding agent aggregate, thereby preparing a detectable antibody-antibody binding agent aggregate.

It must be noted that, as used in the specification and the appended claims, the singular forms “a,” “an,” and “the” include plural referents unless the context clearly dictates otherwise.

As used herein, the term “antibody” refers to an immunoglobulin molecule that includes one or more antigen-binding domains that specifically bind to a particular antigen. In some embodiments, the term encompasses any polypeptide or polypeptide complex that includes immunoglobulin structural elements sufficient to confer specific binding. Exemplary antibodies include, but are not limited to monoclonal antibodies, polyclonal antibodies, and fragments thereof. In some embodiments, an antibody may include one or more sequence elements are humanized, primatized, chimeric, etc., as is known in the art. In many embodiments, the term “antibody” is used to refer to one or more of the art-known or developed constructs or formats for utilizing antibody structural and functional features in alternative presentation. For example, an antibody utilized in accordance with the present invention is in a format selected from, but not limited to, intact IgA, IgG, IgE, or IgM antibodies; bi- or multi-specific antibodies; antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies; cameloid antibodies; masked antibodies; Small Modular ImmunoPharmaceuticals; single chain or Tandem diabodies; VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody is an IgG antibody.

Antibodies are generated by immunizing host animals (e.g., human, dog, cat) with an immunogenic material (e.g., antigen). Antibodies can be produced against a wide range of different antigens. Exemplary antigens include, but are not limited to full-length proteins, protein fragments, peptides, viral proteins, whole organisms (e.g., bacteria), or cells. In some embodiments, an antibody is generated against a full-length antigen. In some embodiments, an antibody is generated against an antigenic epitope. In some embodiments, an antibody is generated against a viral protein. In some embodiments, an antibody is generated against a SARS-COV2 protein. In some embodiments, an antibody is generated against a SARS-COV2 N nucleocapsid protein. In some embodiments, an antibody is generated against a SARS-COV2 S spike protein. In some embodiments, an antibody is a SARS-COV2 antibody. In some embodiments, an antibody is generated against an NMDAR1 peptide. In some embodiments, an antibody is generated against an NMDAR1 P2 peptide. In some embodiments, an antibody is generated against an NMDAR1 protein. In some embodiments, an antibody is an NMDAR1 autoantibody.

As used herein, the term “antibody binding antigen” or “antigen” refers to an agent that binds to an antibody. In some embodiments, an antibody binding antigen binds to an antibody and may or may not induce a particular physiological response in an organism. In general, an antibody binding antigen may be or include any chemical entity such as, for example, a small molecule, a nucleic acid, a polypeptide, a carbohydrate, a lipid, a polymer (including biologic polymers [e.g., nucleic acid and/or amino acid polymers] and polymers other than biologic polymers [e.g., other than a nucleic acid or amino acid polymer]) etc. In some embodiments, an antibody binding antigen is or comprises a polypeptide. In some embodiments, an antibody binding antigen is or comprises a glycan. Those of ordinary skill in the art will appreciate that, in general, an antibody binding antigen may be provided in isolated or pure form, or alternatively may be provided in crude form (e.g., together with other materials, for example in an extract such as a cellular extract or other relatively crude preparation of an antigen-containing source). In some certain embodiments, an antibody binding antigen is present in a cellular context (e.g., an antigen is expressed on the surface of a cell or expressed in a cell). In some embodiments, an antibody binding antigen can be expressed in a cell. In some embodiments, an antibody binding antigen can be produced by expressing the antigen in a cell. In some embodiments, an antibody binding antigen can be expressed in an E. coli cell. In some embodiments, an antibody binding antigen is a recombinant antigen. In some embodiments, the antibody binding agent can be synthesized in vitro. In some embodiments, an antibody binding antigen is a viral protein. In some embodiments, an antibody binding antigen is a SARS-COV2 N protein. In some embodiments, an antibody binding antigen is a SARS-COV2 S protein. In some embodiments, an antibody binding antigen is a SARS-COV2 protein. In some embodiments, an antibody binding antigen is an NMDAR1 peptide. In some embodiments, an antibody binding antigen is an NMDAR1 P2 peptide. In some embodiments, an antibody binding antigen is an NMDAR1 protein. In some embodiments, the antibody binding antigen is a protein or non-protein from human or animal.

As used herein, the term “antibody binding agent” refers to an agent that binds to an antibody. In some embodiments, the antibody binding agent includes an antibody binding antigen and a detectable label. In some embodiments, the antigen binding agent includes a detectable label that is conjugated or otherwise attached to the antibody binding antigen. In some embodiments, the detectable label is conjugated with the antibody binding antigen via cross-linking. In some embodiments, the detectable label is conjugated with the antibody binding antigen via GFP-complementation. In some embodiments, the antibody binding agent can be synthesized in vitro.

As used herein, the term “detectable label” refers to a directly or indirectly detectable moiety that is associated with (e.g., conjugated to) a molecule to be detected (e.g., an antigen). The detectable label can be directly detectable by itself (e.g., radioisotope labels or fluorescent labels) or, in the case of an enzymatic label, can be indirectly detectable, e.g., by catalyzing chemical alterations of a chemical substrate compound or composition, which chemical substrate compound or composition is directly detectable. In some embodiments, detectable labels can be suitable for small scale detection. In some embodiments, detectable labels can be suitable for high-throughput screening. As such, suitable detectable labels include, but are not limited to, radioisotopes, fluorophores, chemiluminescent compounds, bioluminescent compounds, and dyes.

In some embodiments, a detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties.

In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine 0-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™ Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester). In some embodiments, the detectable label is a green fluorescent protein (GFP).

In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, the detectable label is or includes a bioluminescent moiety. Non-limiting examples of bioluminescent moieties can include, but are not limited to, firefly luciferase, Renilla-luciferin 2-monooxygenase, Metridia coelenterazine-dependent luciferase (MetLuc), bacterial luciferase, or Dinoflagellate luciferase. In some embodiments, the bioluminescent moiety can include Gaussia luciferase (Gluc). In some embodiments, the detectable label comprises an enzyme that is detectable with substrate, such as horseradish peroxidase (HRP), or other enzymes.

As used herein, a “biological sample” is obtained from a subject for analysis and generally includes cells and/or other biological material from the subject. In some embodiments, a subject from which a biological sample is obtained can be a healthy or asymptomatic individual, an individual that has or is suspected of having a disease (e.g., COVID-19), and/or an individual that is in need of therapy or suspected of needing therapy.

In some embodiments, the biological sample can be obtained as a tissue sample, such as a tissue section, biopsy, a core biopsy, needle aspirate, or fine needle aspirate. In some embodiments, the biological sample can be a fluid sample, such as a blood sample, urine sample, or saliva sample. In some embodiments, the biological sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample is nose mucus. In some embodiments, the biological sample is mucus from other organs. In some embodiments, the biological sample is a serum. In some embodiments, the biological sample is a serum from a human. In some embodiments, the biological sample is a serum from another animal (e.g., dog, cat, mouse, rat, hamster, monkey, or other non-human primate). In some embodiments, the biological sample can be from an animal, such as a mammal (e.g., human or a non-human simian), or avian (e.g., bird), or other organism. Examples of animals can include, but are not limited to, a mammal such as a rodent, mouse, rat, rabbit, guinea pig, ungulate, horse, sheep, pig, goat, cow, cat, dog, or primate (i.e., human or non-human primate).

### Aggregating Agent

As used herein, the term “aggregating agent” refers to an agent that binds to a molecule (e.g., antibody, protein) and is capable of facilitating the accumulation or formation of tangled clusters, thereby forming an aggregate of the molecules. In some embodiments, an aggregating agent binds to an antibody. In some embodiments, an aggregating agent binds to an antibody binding agent. In some embodiments, an aggregating agent comprises a fusion protein. As used herein, a “fusion protein” refers to a protein produced through the joining of two or more genes that originally coded for separate proteins. Translation of the joined fusion gene results in a single or multiple polypeptides derived from each of the original proteins.

In some embodiments, the fusion protein is a protein-A/G/L fusion protein. In some embodiments, the fusion protein comprises protein A, protein G, or protein A/G or their variants. In some embodiments, the fusion protein comprises multiple copies of protein A, protein G, or protein A/G or their variants. In some embodiments, the fusion protein comprises protein A, protein G, protein A/G, or protein L. In some embodiments, the fusion protein comprises multiple copies of protein A, protein G, protein A/G, or protein L or their variants. In some embodiments, an aggregating agent may not be limited to protein A, G, L.

Protein A, protein G, protein A/G, and protein L are bacterial proteins that bind human IgG, but also IgG from other species. In some embodiments, the proteins are widely used as affinity matrices for purification of IgG. Protein A/G is a recombinant fusion protein that combines IgG binding domains of both protein A and protein G. In some embodiments, protein A/G binds to all subclasses of human IgG, making it useful for purifying polyclonal or monoclonal IgG antibodies. In some embodiments, protein A, protein G, protein A/G, and protein L are all commonly used to purify, immobilize or detect immunoglobulins.

Protein A - amino acid sequence
(SEQ ID NO: 1)
AAQHDEAQQNAFYQVLNMPNLNADQRNGFIQSLKDDPSQSANVLGEAKKL
NESQAPKADNNFNKEQQNAFYEILNMPNLNEEQRNGFIQSLKDDPSQSAN
LLSEAKKLNESQAPKADNKFNKEQQNAFYEILHLPNLNEEQRNGFIQSLK
DDPSQSANLLAEAKKLNDAQAPKADNKFNKEQQNAFYEILHLPNLTEEQR
NGFIQSLKDDPSVSKEILAEAKKLNDAQAPKEEDNNKPGKEDGNKPGKED
GN
Protein G (Immunoglobulin G-binding protein G)-
amino acid sequence
(SEQ ID NO: 2)
MEKEKKVKYFLRKSAFGLASVSAAFLVGSTVFAVDSPIEDTPIIRNGGEL
TNLLGNSETTLALRNEESATADLTAAAVADTVAAAAAENAGAAAWEAAAA
ADALAKAKADALKEFNKYGVSDYYKNLINNAKTVEGVKDLQAQVVESAKK
ARISEATDGLSDFLKSQTPAEDTVKSIELAEAKVLANRELDKYGVSDYHK
NLINNAKTVEGVKDLQAQVVESAKKARISEATDGLSDFLKSQTPAEDTVK
SIELAEAKVLANRELDKYGVSDYYKNLINNAKTVEGVKALIDEILAALPK
TDTYKLILNGKTLKGETTTEAVDAATAEKVFKQYANDNGVDGEWTYDDAT
KTFTVTEKPEVIDASELTPAVTTYKLVINGKTLKGETTTEAVDAATAEKV
FKQYANDNGVDGEWTYDDATKTFTVTEKPEVIDASELTPAVTTYKLVING
KTLKGETTTKAVDAETAEKAFKQYANDNGVDGVWTYDDATKTFTVTEMVT
EVPGDAPTEPEKPEASIPLVPLTPATPIAKDDAKKDDTKKEDAKKPEAKK
EDAKKAETLPTTGEGSNPFFTAAALAVMAG AGALAVASKR KED
Protein A/G - amino acid sequence
(SEQ ID NO: 3)
NAAQHDEAQQNAFYQVLNMPNLNADQRNGFIQSLKDDPSQSANVLGEAQK
LNDSQAPKADAQQNNFNKDQQSAFYEILNMPNLNEAQRNGFIQSLKDDPS
QSTNVLGEAKKLNESQAPKADNNFNKEQQNAFYEILNMPNLNEEQRNGFI
QSLKDDPSQSANLLSEAKKLNESQAPKADNKFNKEQQNAFYEILHLPNLN
EEQRNGFIQSLKDDPSQSANLLAEAKKLNDAQAPKADNKFNKEQQNAFYE
ILHLPNLTEEQRNGFIQSLKDDPSVSKEILAEAKKLNDAQAPKEEDSLEG
SGSGTYKLILNGKTLKGETTTEAVDAATAEKVFKQYANDNGVDGEWTYDD
ATKTFTVTEKPEVIDASELTPAVTTYKLVINGKTLKGETTTKAVDAETAE
KAFKQYANDNGVDGVWTYDDATKTFTVTE
Protein L - amino acid sequence
(SEQ ID NO: 4)
MKINKKLLMAALAGAIVVGGGANAYAAEEDNTDNNLSMDEISDAYFDYHG
DVSDSVDPVEEEIDEALAKALAEAKETAKKHIDSLNHLSETAKKLAKNDI
DSATTINAINDIVARADVMERKTAEKEEAEKLAAAKETAKKHIDELKHLA
DKTKELAKRDIDSATTINAINDIVARADVMERKTAEKEEAEKLAAAKETA
KKHIDELKHLADKTKELAKRDIDSATTIDAINDIVARADVMERKLSEKET
PEPEEEVTIKANLIFADGSTQNAEFKGTFAKAVSDAYAYADALKKDNGEY
TVDVADKGLTLNIKFAGKKEKPEEPKEEVTIKVNLIFADGKTQTAEFKGT
FEEATAKAYAYADLLAKENGEYTADLEDGGNTINIKFAGKETPETPEEPK
EEVTIKVNLIFADGKIQTAEFKGTFEEATAKAYAYANLLAKENGEYTADL
EDGGNTINIKFAGKETPETPEEPKEEVTIKVNLIFADGKTQTAEFKGTFE
EATAEAYRYADLLAKVNGEYTADLEDGGYTINIKFAGKEQPGENPGITID
EWLLKNAKEEAIKELKEAGITSDLYFSLINKAKTVEGVEALKNEILKAHA
GEETPELKDGYATYEEAEAAAKEALKNDDVNNAYEIVQGADGRYYYVLKI
EVADEEEPGEDTPEVQEGYATYEEAEAAAKEALKEDKVNNAYEVVQGADG
RYYYVLKIEDKEDEQPGEEPGENPGITIDEWLLKNAKEDAIKELKEAGIS
SDIYFDAINKAKTVEGVEALKNEILKAHAEKPGENPGITIDEWLLKNAKE
AAIKELKEAGITAEYLFNLINKAKTVEGVESLKNEILKAHAEKPGENPGI
TIDEWLLKNAKEDAIKELKEAGITSDIYFDAINKAKTIEGVEALKNEILK
AHKKDEEPGKKPGEDKKPEDKKPGEDKKPEDKKPGEDKKPEDKKPGKTDK
DSPNKKKKAKLPKAGSEAEILTLAAAALSTAAGAYVSLKKRK

In some embodiments, an aggregate can be detected by imaging the biological sample. An aggregate in a biological sample can be identified using a variety of different imaging techniques, e.g., expansion microscopy, bright field microscopy, dark field microscopy, phase contrast microscopy, electron microscopy, fluorescence microscopy, reflection microscopy, interference microscopy, confocal microscopy, and visual identification (e.g., by eye), and combinations thereof. In some embodiments, an aggregate can be detected by using fluorescent microscopy. In some embodiments, an aggregate can be detected by using 470 nm blue LED light. In some embodiments, an aggregate can be detected by any imaging method known in the art.

In some embodiments, an aggregate can be detected within 5 minutes (e.g., within 4 minutes, within 3 minutes, within 2 minutes, or within 1 minute) of adding the aggregating agent to the biological sample. In some embodiments, an aggregate can be detected about a week (e.g., about 6 days, about 5 days, about 4 days, about 3 days, about 2 days, or about 1 day) after adding the aggregating agent to the biological sample.

### Kits

A “kit,” as used herein, typically includes a package or an assembly including one or more of the compositions or devices of the invention, and/or other compositions or devices associated with the invention, as previously described. Each of the compositions of the kit, if present, may be provided in liquid form (e.g., in solution), or in solid form (e.g., a dried powder). In certain embodiments, one or more of the compositions may be constitutable or otherwise processable (e.g., to an active form), for example, by the addition of a suitable solvent or other species, which may or may not be provided with the kit. A kit may further include other compositions or components associated with the invention include, but are not limited to, solvents, surfactants, diluents, salts, buffers, emulsifiers, chelating agents, fillers, antioxidants, binding agents, bulking agents, preservatives, drying agents, packaging materials, tubes, bottles, filters, containers, tapes, or adhesives. A kit may include instructions in any form that are provided in connection with the compositions of the invention in such a manner that one of ordinary skill in the art would recognize that the instructions are to be associated with the compositions of the invention. The instructions may be provided in any form recognizable by one of ordinary skill in the art as a suitable vehicle for containing such instructions. For example, written or published, verbal, audible (e.g., telephonic), digital, optical, visual (e.g., videotape, DVD, etc.) or electronic communications (including Internet or web-based communications), provided in any manner.

Provided herein are kits including (a) an antibody binding agent, wherein the antibody binding agent comprises an antibody binding antigen and a detectable label; (b) an aggregating agent; and (c) instructions to add the antibody binding agent and the aggregating agent to a biological sample to generate an antibody-antibody binding agent aggregate.

In some embodiments, the antibody binding antigen is a SARS-COV2 protein. In some embodiments, the SARS-COV2 protein is a SARS-COV2 N nucleocapsid protein. In some embodiments, the SARS-COV2 protein is a SARS-COV2 spike protein. In some embodiments, the antibody binding antigen is an NMDAR1 peptide. In some embodiments, the antibody binding antigen is an NMDAR1 P2 peptide. In some embodiments, the antibody binding antigen is an NMDAR1 protein. In some embodiments, the antibody binding antigen is a protein or non-protein from human or animal.

In some embodiments, the detectable label comprises a fluorophore. In some embodiments, the fluorophore comprises a green fluorescent protein (GFP). In some embodiments, the detectable label comprises a bioluminescent label. In some embodiments, the bioluminescent label comprises Gaussia luciferase (Gluc). In some embodiments, the detectable label is conjugated with the antibody binding antigen via cross-linking. In some embodiments, the detectable label is conjugated with the antibody binding antigen via GFP-complementation.

In some embodiments, the aggregating agent comprises a fusion protein. In some embodiments, the fusion protein is a protein-A/G/L fusion protein. In some embodiments, the fusion protein comprises protein-A, protein-G, protein-A/G, or variants thereof.

In some embodiments, the instructions comprise instructions to image the biological sample.

### EXAMPLES

The disclosure is further described in the following examples, which do not limit the scope of the disclosure described in the claims.

Production of scN-GFP in Escherichia coli

Nucleocapsid protein sequence of SARS-CoV2 virus was from NCBI reference sequence database (Accession NP 828858). Gene encoding scN-GFP fusion protein with a 6His tag was synthesized and cloned into pET-21d vector. BL21(DE3)pLysS competent Escherichia coli cells were purchased from EMD (cat. 70236-3) for transformation of the plasmid. In brief, a single E. coli colony was inoculated in 2 ml of LB medium containing 100 μg/ml carbenicillin. After overnight shaking at 37° C., the E. coli culture was diluted 1:10 with LB medium containing both 100 μg/ml carbenicillin and 0.2 mM IPTG. The diluted E. coli culture was vigorously shaked for 4 h at 37° C. to induce over-expression of scN-GFP proteins. After centrifugation, E. coli cell pellet was suspended in 1×PBS, 0.25 M NaCl, 1 mM PMSF, and then sonicated on ice. scN-GFP proteins in the supernatant were collected after centrifugation. HisPur Ni-NTA resins (cat. 88221; ThermoFisher Scientific, Waltham, MA, USA) were washed with 1×PBS before loaded with the scN-GFP containing supernatant. After 10× volume of washing with 1×PBS, 0.25 M NaCl, scN-GFP proteins were eluted with 1×PBS, 0.25 M NaCl, 120 mM imidazole.

### One-Step Test

Rabbit polyclonal antibodies against SARS-CoV2 N nucleocapsid proteins were purchased from SinoBiological (cat. 40588-T62). Protein A/G/L was purchased from Novus Biologicals (NBP2-34985) and diluted to 1 ug/ul with antibody diluent solution (S080983-2; DAKO, Denmark). Protein A/G/L tagged with 6His was also over-expressed in E. coli and purified in the laboratory. The purified scN-GFP proteins were diluted with 1×PBS, 0.25 M NaCl for use. One μl of serum was mixed with 3 μl of the diluted scN-GFP and 1 ul of protein A/G/L (1 ug/ul). After incubation for 5 min at room temperature, antigen-antibody aggregates of the one-step test were examined for GFP green fluorescence using microscope EVOS FL (ThermoFisher Scientific, Waltham, MA, USA).

### Human Serum Samples

Forty serum samples from SARS-CoV2 patients diagnosed by PCR or antigen tests were purchased from RayBiotech. Blood from 37 patients were drawn 33-35 days after the diagnostic test. Blood of patient A11, A18, and B7 were drawn 64, 25, and 18 days after the test, respectively. Fifty pre-pandemic human serum samples were purchased from San Diego Blood Bank. The studies were approved by Human Research Protections Program at University of California San Diego.

### Example 1—Detection of Antibodies Against SARS-COV2 N Proteins

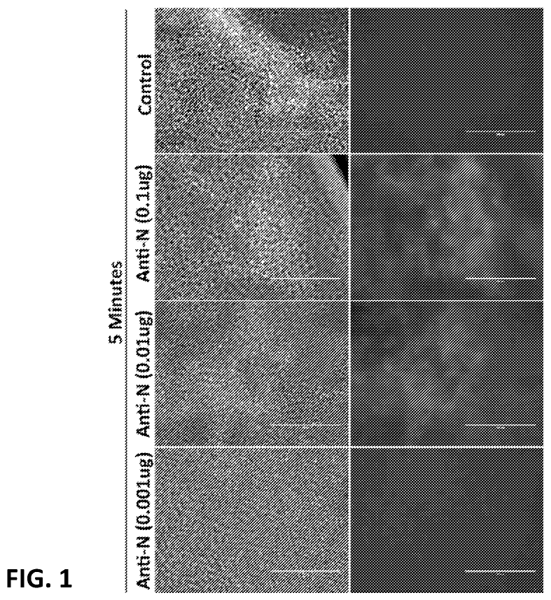

GFP-N fusion proteins were generated from E. coli protein expression. Rabbit antibodies against SARS-COV2 N proteins were purchased commercially. One microliter of human serum was used as the negative control, whereas 100 ng, 10 ng, and 1 ng of rabbit anti-SARS-COV2 N antibodies were diluted into one microliter of human serum as the positive controls. One microliter of human serum with or without the rabbit anti-N antibodies was mixed with the GFP-N proteins in the presence of protein-A/G/L fusion proteins. 5 minutes later, the aggregates were visualized under fluorescence microscope (FIG. 1).

No green fluorescence was detected from the aggregates of the negative control human serum. Green fluorescence was readily observed in the aggregates of the human serum containing anti-SARS-COV2 N antibodies at an amount of 100 ng and 10 ng, respectively. Green fluorescence was undetectable in the aggregates of the human serum containing 1 ng of anti-SARS-COV2 N antibodies, suggesting that it is possible to detect 10 ng of the antibodies that is equivalent to detection of ˜0.1% of human blood antibodies per microliter of human blood.

### Example 2—Robustness of Detection Assay

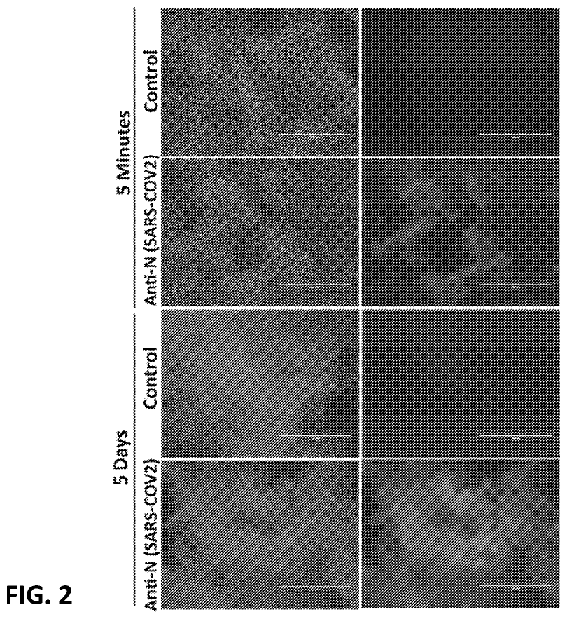

To examine whether a prolonged incubation may increase non-specific background in the aggregates of the negative control reaction, the aggregates in the same set of the negative and the positive reactions were examined after 5 minutes and 5 days, respectively (FIG. 2).

There was no non-specific green fluorescence background in the aggregates of the negative control human serum after 5 minutes incubation of the mixture. These aggregates displayed no increase of non-specific green fluorescence background even after 5 days incubation. In contrast, green fluorescence intensities of the aggregates from the human serum containing anti-SARS-COV2 N antibodies (100 ng) remained very high from 5 minutes to 5 days.

In conclusion, there was no non-specific fluorescence background in the aggregates from the negative control human serum even after 5 days incubation. On the other hand, the GFP green fluorescence intensities of the aggregates from the human serum containing anti-SARS-COV2 antibodies remained very high and stable, supporting the robustness of the detection assay.

### Example 3—Detection of Blood Neutralizing Antibodies Against SARS-COV2 from COVID19 Patients

To detect neutralizing antibodies against the SARS-CoV2, receptor binding domain (RBD) of the SARS-CoV2 spike protein is fused with GFP. The fusion protein is produced and purified from E. coli as described above. Both pre-pandemic human sera from San Diego Blood Bank (SDBB) and the sera from recovered COVID19 patients were purchased, wherein 5 representative pre-pandemic samples from San Diego Blood Bank (SDBB1-5) were negative, and 5 representative samples of the recovered COVID19 patients (P1-5) were positive. The GFP green fluorescence intensities indicated the titers of the neutralizing antibodies (FIG. 3).

### Example 4—Detection of Blood Neutralizing Antibodies from Vaccination

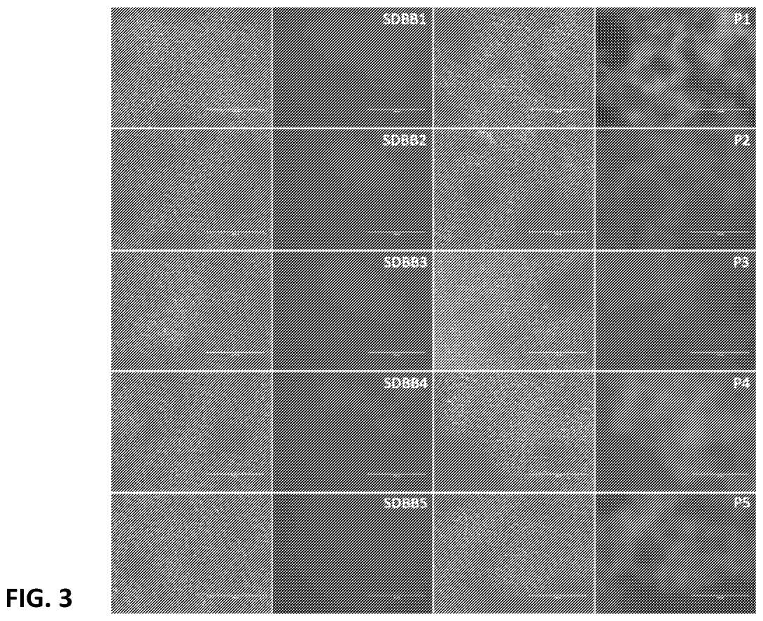

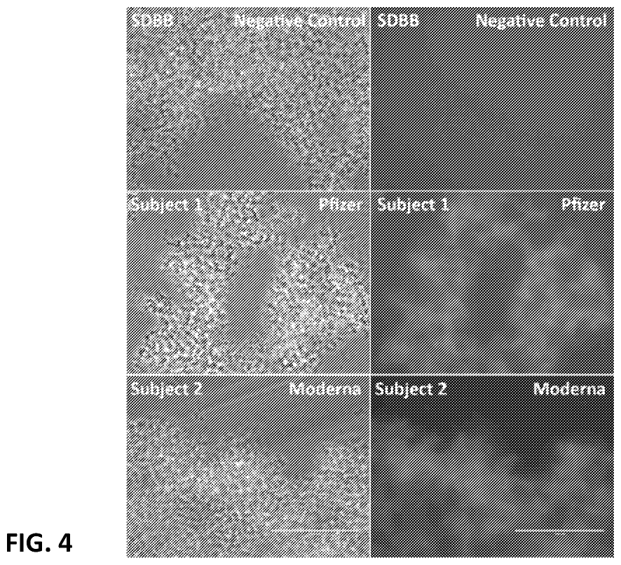

Blood serum was collected from subjects that have been vaccinated with vaccines from either Pfizer or Moderna. 10 to 14 days after the second shot, neutralizing antibodies produced in blood by the vaccination were examined. The pre-pandemic blood sample from San Diego Blood Bank was used as a negative control. Subjects vaccinated with either Pfizer of Moderna vaccines produced neutralizing antibodies against the RBD of the SARS-CoV2 spike protein (FIG. 4).

### Example 5 Detection of Neutralizing Antibodies in Nose Mucus

Unlike the neutralizing antibodies in blood, the neutralizing antibodies in nose mucus provide the first defense against SARS-CoV2 infection. Therefore, they are more important for virus community transmission. In addition, this detection is noninvasive.

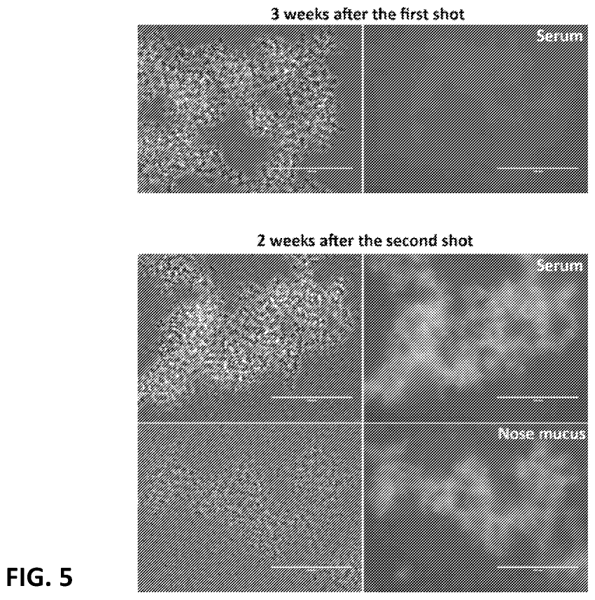

The presence of the neutralizing antibodies was examined in nose mucus from a subject two weeks after the second shot of Moderna vaccine. There were little neutralizing antibodies in blood 3 weeks after the first shot, but a large amount of the neutralizing antibodies were present in blood two weeks after the second shot. A large amount of the neutralizing antibodies were also present in nose mucus (FIG. 5).

### Example 6—Detection of Anti-NMDAR1 Antibodies Against NMDAR1 P2 Antigenic Epitope in Mouse Blood Serum

An antigenic epitope P2 (20 amino acid residues) of mouse NMDAR1 protein was used to immunize mice to generate antibodies against the NMDAR1 antigenic epitope. The production of anti-NMDAR1 antibodies in mouse blood was confirmed with immunohistochemistry. The same NMDAR1 P2 peptide antigen was labelled with GFP using E. coli protein over-expression. One microliter of mouse blood serum was used for the detection of anti-NMDAR1 antibodies in mice immunized with the P2 peptide antigens. The control mice were immunized without the P2 peptide antigens.

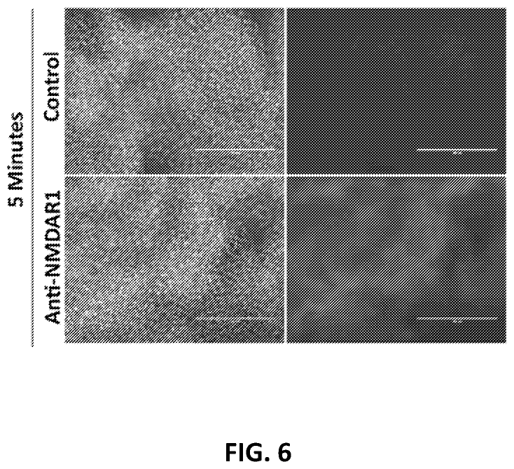

No green fluorescence in the aggregates from the blood serum of the negative control mice was observed, whereas green fluorescence was readily observed in −5 minutes in the aggregates from the mouse blood serum containing anti-NMDAR1 antibodies against the P2 peptide antigen (FIG. 6).

### Example 6—Development of a One-Step Assay for Detection of Other Antibodies

Many human autoimmune diseases are caused by autoantibodies. For example, anti-NMDAR1 autoantibodies have been shown to cause anti-NMDAR1 encephalitis. Additionally, immunotherapy has been used to treat many human diseases. It would be beneficial to monitor the dynamics of these antibodies in blood using a simple quick assay.

Anti-NMDAR1 autoantibodies were detected in mice immunized with NMDAR1 peptide antigens. The presence of the anti-NMDAR1 autoantibodies was first validated by immunohistochemical analysis. The results from the One-Step quick assay was 100% consistent with the immuohistochemical detection of mice carrying anti-NMDAR1 autoantibodies.

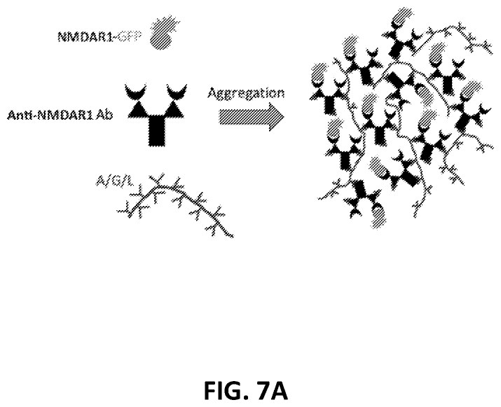

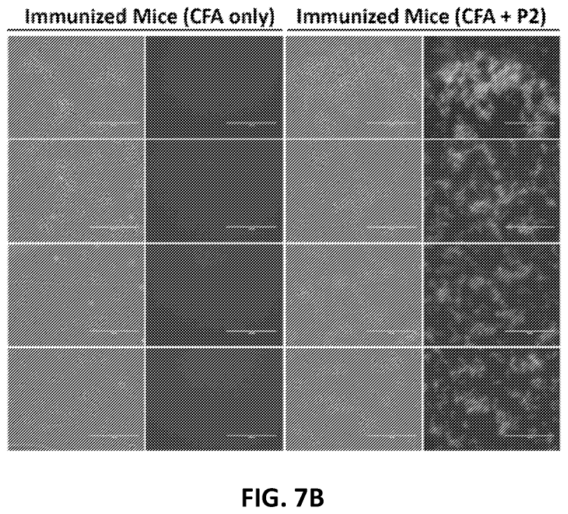

The One-Step strategy is illustrated in FIG. 7A. Four mice were immunized with Complete Freund Adjuvant (CFA) only, wherein these mice were negative controls. Another group of four mice were immunized with CFA and NMDAR1 P2 peptide and produced anti-NMDAR1 autoantibodies that were readily detected by the One-Step assay (FIG. 7B).

### Example 7—Development of One-Step Assay Using Other Detectable Labels

Many detection labels other than GFP can be conjugated with antigens for antibody detection after aggregation. Gaussia luciferase (Gluc) was fused to SARS-CoV2 N nucleocapsid protein for detection of SARS-CoV2 infection. The advantage of using the Gaussia luciferase is that there is no need for fluorescence microscope for the assay. The N-Gluc fusion proteins were produced and purified from E. coli.

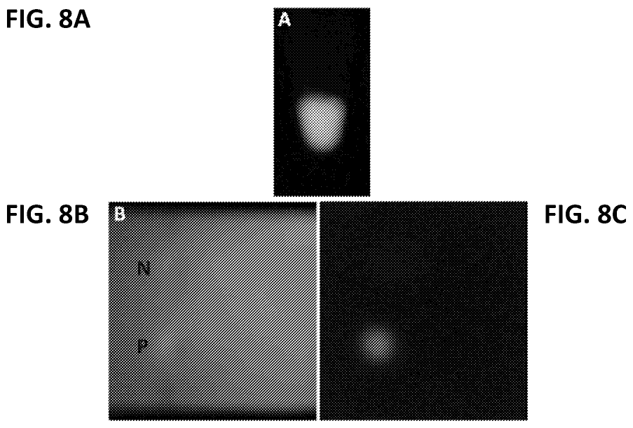

In FIG. 8A, the N-Gluc fusion proteins were shown to have strong luciferase activities (emitting blue light in dark) when incubated with substrate coelenterazine in an eppendorf tube.

The One-step assay was conducted with a slight modification. The assay mixtures were spotted on Whatman paper for paper chromatography using coelenterazine solution, wherein the chromatography took about 1 minute. The antibody aggregates will stay on the initial spots. Therefore, if antibodies bind SARS-CoV2 N nucelocapsid proteins, the N-Gluc will be trapped in aggregates on the spot. In FIG. 8B, a negative control serum spot (N) and a COVID19 patient serum spot (P) are shown in the image taken under weak light. The P spot emitted blue light. When the image was taken in dark (FIG. 8C), it was clear that only the P spot is positive for anti-SARS-CoV2 N antibodies. This method can be used to detect other antibodies for home use without need of any special device or equipment.

### Example 8—Development of the One-Step Assay for Detection of Antibodies Against SARS-COV2 N Nucleocapsid Protein

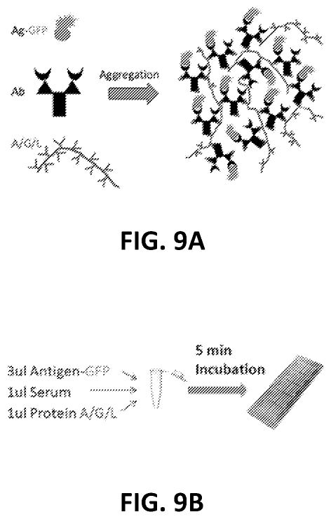

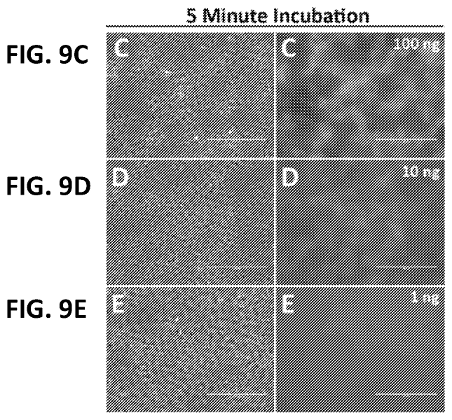

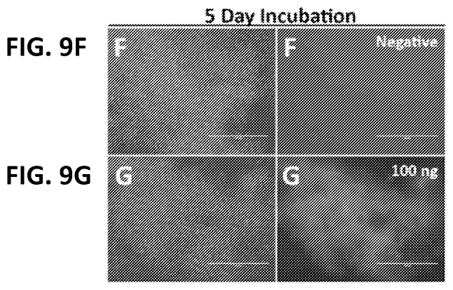

Protein A/G/L consisted of five IgG-binding regions of protein A, two IgG-binding regions of protein G, and five light chain-binding regions of protein L. In a mixture of antibodies and fluorescence-labeled antigens, addition of protein A/G/L would instantly cross-link antigen-antibody molecules into large aggregates by binding heavy and/or light chain of antibodies (FIGS. 9A-9B). Such large aggregates would emit strong fluorescence. Here, SARS-CoV2 N nucleocapsid protein (scN) was fused to green fluorescence protein (GFP) with a 6His tag, over-expressed in BL21(DE3) E. coli cells, and purified with Ni-NTA resins. Rabbit polyclonal antibodies against SARS-CoV2 N nucleocapsid protein (cat. 40588-T62; SinoBiological, Beijing, China) were diluted in human serum to a concentration of 100 ng/ul, 10 ng/ul, 1 ng/ul, respectively. The scN-GFP proteins were diluted at −30 ng/ul and incubated with the diluted rabbit antibodies and protein A/G/L (FIGS. 9C-9E). After 5 min incubation at room temperature, the reactions were loaded into capillary slides (cat. 76237-746; VWR, Radnor, Pennsylvania, USA) for examination. As expected, antigen-antibody aggregates emitted strong green fluorescence with an input of 100 ng of the rabbit antibodies. The limit of detection appears to be −10 ng of the rabbit antibodies.

To examine non-specific background of antigen-antibody aggregates, a mixture of control human serum, scN-GFP, and protein A/G/L was incubated for 5 days at 4° C. (FIGS. 9F-9G). No fluorescence background was observed in the antibody aggregates from the control human serum, whereas strong fluorescence persisted in the antigen-antibody aggregates from human serum containing the rabbit antibodies against SARS-CoV2 N proteins. These data suggested that the one-step quick test may have a high sensitivity and specificity.

### Example 9—Sensitivity and Specificity of the One-Step Assay in Patient Serum Samples

To examine whether the one-step test can detect anti-scN antibodies in patients with SARS-CoV2 infection, 40 serum samples of patients diagnosed by PCR were purchased from RayBiotech. Patient blood were drawn 33-35 days after the diagnostic test, except for 3 patients whose blood were drawn 64, 25, and 18 days after the test, respectively. These samples had been studied by RayBiotech for antibodies against SARS-CoV2 N proteins using LFIA rapid antibody test. Samples were deemed positive if either IgG or IgM is positive by LFIA, and 20 out of the 40 samples were positive for anti-scN antibodies. The other 20 samples were negative by LFIA. 50 pre-pandemic human serum samples from San Diego Blood Bank were used as negative controls.

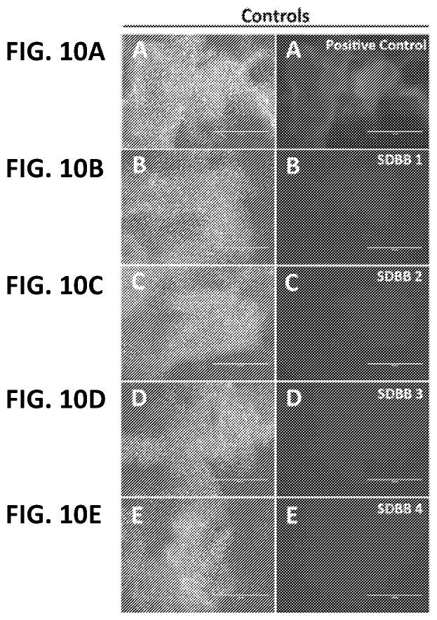

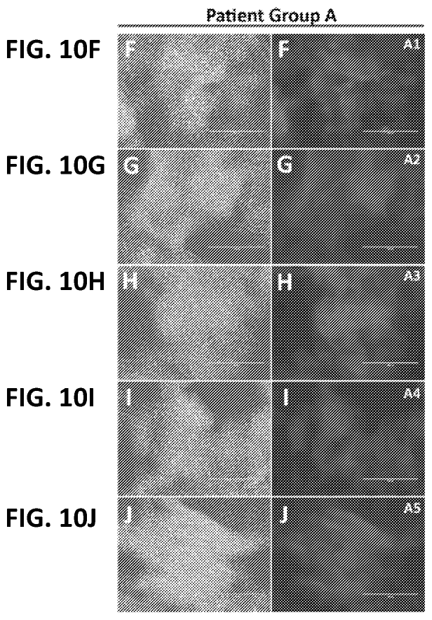

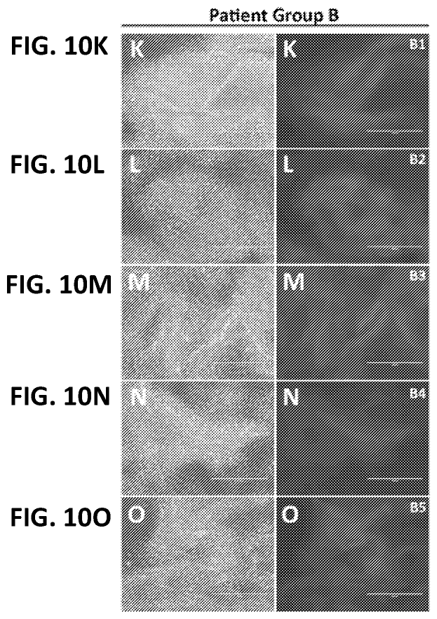

The one-step assay was conducted to examine anti-scN antibodies in all of the 90 serum samples. A total of 39 positive serum samples were identified, and all of them came from the 40 SARS-CoV2 patients. None of the 50 pre-pandemic serum samples were positive for anti-scN antibodies (FIGS. 10A-10E, Controls). All of the 20 patient samples deemed positive by LFIA were also identified as positive by the one-step test (FIGS. 10F-10J, Group A). Out of the 20 patient samples deemed negative by LFIA, 19 samples were identified as positive by the one-step test (FIGS. 10K-10O, Group B). These results are summarized in Table 1.

TABLE 1
Comparison between LFIA rapid antibody
test and the one-step test.

| | RayBiotech patients | | |
|---|---|---|---|
| | Group A | Group B | SDBB pre-pandemic |
| | (n = 20) | (n = 20) | controls (n = 50) |
| Age (SD) | 64.3 (16.1) | 64.1 (17) | 52.5 (17.9) |
| Positives by LFIA | 20 | 0 | N/A |
| Positives by One-Step | 20 | 19 | 0 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preparing a detectable antibody-antibody binding agent aggregate, the method comprising:
(a) contacting a biological sample with an antibody binding agent, wherein the antibody binding agent comprises an antibody binding antigen and a detectable label, wherein the antibody binding agent binds an antibody in the biological sample;
(b) adding an aggregating agent to the biological sample, wherein the aggregating agent binds to the antibody and forms an antibody-antibody binding agent aggregate; and
(c) detecting a signal from the detectable label of the antibody binding agent associated with the antibody-antibody binding agent aggregate, thereby preparing a detectable antibody-antibody binding agent aggregate.

2. (canceled)

3. (canceled)

4. (canceled)

5. The method of claim 1, wherein the antibody is a SARS-COV2 antibody.

6. (canceled)

7. (canceled)

8. (canceled)

9. (canceled)

10. (canceled)

11. (canceled)

12. The method of claim 1, wherein the antibody binding antigen is a SARS-COV2 protein.

13. (canceled)

14. (canceled)

15. The method of claim 1, wherein the detectable label comprises a fluorophore.

16. (canceled)

17. (canceled)

18. (canceled)

19. (canceled)

20. The method of claim 1, wherein the detectable label is conjugated with the antibody binding antigen via cross-linking.

21. The method of claim 1, wherein the detectable label is conjugated with the antibody binding antigen via GFP-complementation.

22. The method of claim 1, wherein the aggregating agent comprises a fusion protein.

23. The method of claim 22, wherein the fusion protein is a protein-A/G/L fusion protein.

24. The method of claim 22, wherein the fusion protein comprises protein-A, protein-G, protein-A/G, or variants thereof.

25. (canceled)

26. (canceled)

27. The method of claim 1, wherein the detecting comprises imaging using 470 nm blue LED light.

28. The method of claim 1, wherein the antibody binding agent is produced by expressing the antibody binding agent in a cell.

29. (canceled)

30. The method of claim 1, wherein the antibody-antibody binding agent aggregate is detected within 5 minutes of adding the aggregating agent to the biological sample.

31. The method of claim 1, wherein the antibody-antibody binding agent aggregate can be detected up to 5 days after adding the aggregating agent to the biological sample.

32. A kit comprising:
(a) an antibody binding agent, wherein the antibody binding agent comprises an antibody binding antigen and a detectable label;
(b) an aggregating agent; and
(c) instructions to add the antibody binding agent and the aggregating agent to a biological sample to generate an antibody-antibody binding agent aggregate.

33. The kit of claim 32, wherein the antibody binding antigen is a SARS-COV2 protein.

34. (canceled)

35. (canceled)

36. The kit of claim 32, wherein the detectable label comprises a fluorophore.

37. (canceled)

38. (canceled)

39. (canceled)

40. The kit of claim 32, wherein the detectable label is conjugated with the antibody binding antigen via cross-linking.

41. The kit of claim 32, wherein the detectable label is conjugated with the antibody binding antigen via GFP-complementation.

42. (canceled)

43. The kit of claim 32, wherein the aggregating agent is a protein-A/G/L fusion protein.

44. The kit of claim 32, wherein the aggregating agent comprises protein-A, protein-G, protein-A/G, or variants thereof.

45. (canceled)


SEQUENCE LISTING (4 sequences)

SEQ ID NO: 1 - protein, 252 aa, Staphylococcus aureus
AAQHDEAQQNAFYQVLNMPNLNADQRNGFIQSLKDDPSQSANVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGN

SEQ ID NO: 2 - protein, 593 aa, Streptococcus sp. group G
MEKEKKVKYFLRKSAFGLASVSAAFLVGSTVFAVDSPIEDTPIIRNGGELTNLLGNSETTLALRNEESATADLTAAAVADTVAAAAAENAGAAAWEAAAAADALAKAKADALKEFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKEDAKKAETLPTTGEGSNPFFTAAALAVMAGAGALAVASKRKED

SEQ ID NO: 3 - protein, 429 aa, Escherichia coli
NAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDSLEGSGSGTYKLILNGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTE

SEQ ID NO: 4 - protein, 992 aa, Finegoldia magna
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYRYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK